(19) United States
(12) Patent Application Publication
(10) Pub. No.: US 2026/0327168 A1
(43) Pub. Date: Oct. 22, 2026

(54) COMPOSITION, COMPOSITION-CONTAINING CONTAINER, SYSTEM, AND COMPOSITION PRODUCTION METHOD

(71) Applicant: AGC INC., Tokyo (JP)

(72) Inventor: Hikaru IWASAKI, Tokyo (JP)

(73) Assignee: AGC INC., Tokyo (JP)

(21) Appl. No.: 19/697,132

(22) Filed: Jun. 3, 2026

Related U.S. Application Data
(63) Continuation of application No. PCT/JP2024/040192, filed on Nov. 12, 2024.

(30) Foreign Application Priority Data
Dec. 5, 2023 (JP) 2023-205627

(51) Int. Cl. C07C 21/18; B01J 23/89; C07C 19/10; C07C 23/06

(52) U.S. Cl. CPC C07C 21/18; B01J 23/8926; C07C 19/10; C07C 23/06

(57) ABSTRACT

A composition contains chlorotrifluoroethylene and hexafluorocyclobutene. The content of the chlorotrifluoroethylene is 8% by mass or more with respect to a total amount of organic compounds contained in the composition, and the content of the hexafluorocyclobutene is 1 ppm by mass or more with respect to the total amount of organic compounds contained in the composition.

### CROSS-REFERENCE TO RELATED APPLICATIONS

This application is a Continuation Application of International Application No. PCT/JP2024/040192, filed Nov. 12, 2024, the disclosure of which is incorporated herein by reference in its entirety. Further, this application claims priority from Japanese Patent Application No. 2023-205627, filed Dec. 5, 2023, the disclosure of which is incorporated herein by reference in its entirety.

### TECHNICAL FIELD

The present disclosure relates to a composition, a composition-containing container, a system, and a composition production method.

### BACKGROUND ART

Chlorotrifluoroethylene is considered to be used as a monomer for various polymers, a precursor for trifluoroethylene, or the like. Trifluoroethylene has a low global warming potential and a low boiling point, and is therefore expected to be a kind of high-pressure refrigerant. Hereinafter, chlorotrifluoroethylene is also referred to as “CTFE,” and trifluoroethylene is also referred to as “HFO-1123.”

Known methods of producing CTFE include a zinc dechlorination method of 1,1,2-trichloro-1,2,2-trifluoroethane, as well as a hydrogen reduction method of 1,1,2-trichloro-1,2,2-ltertrifluoroethane using a catalyst (see, for example, Patent Literature 1). Hereinafter, 1,1,2-trichloro-1,2,2-trifluoroethane is also referred to as “CFC-113.”

### CITATION LIST

### Patent Literature

Patent Literature 1: Chinese Patent Application Publication No. 112657508

### SUMMARY OF INVENTION

### Technical Problem

Compositions obtained by producing CTFE by the hydrogen reduction method of CFC-113 contain components other than CTFE, such as unreacted CFC-113, side reaction products, and products of a further reaction of CTFE.

When a composition containing CTFE is placed in contact with a metal, the pH of the composition may decrease due to the generation of an acid. Examples of a state in which the composition is in contact with a metal include a state in which the composition is accommodated in a container that is at least partially formed of a metal material, and a state in which the composition is retained in a flow path that is at least partially formed of a metal material.

An object of the disclosure is to provide a composition containing chlorotrifluoroethylene (CTFE) that is suppressed from having a decrease in pH when left in contact with a metal for a long period of time, a composition-containing container including the composition, a system containing the composition, and a method of producing the composition.

### Solution to Problem

The disclosure includes the following aspects.

<1> A composition, containing chlorotrifluoroethylene and hexafluorocyclobutene, wherein:
a content of the chlorotrifluoroethylene is 8% by mass or more with respect to a total amount of organic compounds contained in the composition, and
a content of the hexafluorocyclobutene is 1 ppm by mass or more with respect to the total amount of organic compounds contained in the composition.

<2> The composition according to <1>, wherein the content of the hexafluorocyclobutene is 50 ppm by mass or more with respect to the total amount of organic compounds contained in the composition.

<3> The composition according to <1> or <2>, further containing 1,2-dichloro-1,2,3,3,4,4-hexafluorocyclobutane,
wherein a content of the 1,2-dichloro-1,2,3,3,4,4-hexafluorocyclobutane is 1 ppm by mass or more with respect to the total amount of organic compounds contained in the composition.

<4> The composition according to <1> or <2>, further containing 1,1,2-trichloro-1,2,2-trifluoroethane.

<5> The composition according to <3>, further containing 1,1,2-trichloro-1,2,2-trifluoroethane.

<6> The composition according to any one of <1> to <5>, further containing trifluoroethylene.

<7> The composition according to <6>, wherein a content of the trifluoroethylene is 0.3% by mass or more with respect to the total amount of organic compounds contained in the composition.

<8> The composition according to any one of <1> to <7>, further containing water,
wherein a content of the water is 5 ppm by mass or more with respect to a total amount of the composition.

<9> A composition-containing container, including:
the composition according to any one of <1> to <8>; and
a container that accommodates the composition, in which a member that contacts the composition is at least partially formed of a metal material.

<10> A system, including:
the composition according to any one of <1> to <8>; and
a flow path that allows the composition to pass therethrough, in which a member that contacts the composition is at least partially formed of a metal material.

<11> A method of producing the composition according to any one of <1> to <8>, including:
contacting a mixture containing 1,1,2-trichloro-1,2,2-trifluoroethane and hydrogen with a catalyst containing copper and at least one of palladium or platinum.

<12> The method of producing a composition according to <11>, wherein a temperature of the mixture when contacting the mixture with the catalyst is from 150 °C to 300 °C.

<13> The method of producing a composition according to <11> or <12>, wherein the catalyst is a two-component catalyst composed of copper and either palladium or platinum.

<14> The method of producing a composition according to any one of <11> to <13>, wherein a content of hydrogen in the mixture is from 1 to 2.5 times a content of the 1,1,2-trichloro-1,2,2-trifluoroethane on a volume basis.

### Advantageous Effects of Invention

According to the disclosure, a composition containing chlorotrifluoroethylene (CTFE) that is suppressed from having a decrease in pH when left in contact with a metal for a long period of time, a composition-containing container including the composition, a system containing the composition, and a method of producing the composition are provided.

### DESCRIPTION OF EMBODIMENTS

Hereinafter, embodiments of the disclosure are described. These descriptions and examples are intended to illustrate the embodiments and do not limit the scope of the embodiments.

In the disclosure, a numerical range indicated using “to” means a range that includes the numerical values before and after “to” as the minimum and maximum values, respectively.

In the numerical ranges described in stages in the disclosure, the upper or lower limit value of a given numerical range may be replaced with the upper or lower limit value of another numerical range described in stages. Furthermore, within the numerical ranges described in the disclosure, the upper or lower limit value of a given numerical range may be replaced with a value shown in the examples.

In the disclosure, a combination of two or more preferred aspects is a more preferred aspect.

In the disclosure, when there are a plurality of substances corresponding to each component, the amount of each component means the total amount of the plurality of substances unless otherwise specified.

### [Composition]

A composition according to one embodiment of the disclosure contains chlorotrifluoroethylene (CTFE) and hexafluorocyclobutene, which has a CTFE content of 8% by mass or more with respect to a total amount of organic compounds contained in the composition, and a hexafluorocyclobutene content of 1 ppm by mass or more with respect to the total amount of organic compounds contained in the composition. Hereinafter, hexafluorocyclobutene is also referred to as “HFCB.”

As mentioned above, when a composition containing CTFE is placed in contact with a metal, the pH of the composition may decrease due to the generation of an acid. The reason that the pH of the composition decreases is unclear, but it is presumed that as the composition continues to be in contact with the metal, the CTFE contained in the composition gradually dimerizes, and chlorine is eliminated, resulting in the generation of hydrogen chloride.

In contrast, the composition of the embodiment contains 8% by mass or more of CTFE with respect to the total amount of organic compounds, and further contains 1 ppm by mass or more of HFCB with respect to the total amount of organic compounds, thereby suppressing a decrease in pH when left in contact with a metal for a long period of time. The reason for this is unclear, but it is speculated as follows.

CTFE is converted 1,2-dichloro-1,2,3,3,4,4-hexafluorocyclobutane by dimerization, and then converted into HFCB by elimination of chlorine. Since this reaction is an equilibrium reaction, because the initial composition contains HFCB at the above-described content, the equilibrium is less likely to shift in the direction of further production of HFCB. In other words, it is presumed that the decrease in pH is suppressed by suppressing the elimination of chlorine. Hereinafter, 1,2-dichloro-1,2,3,3,4,4-hexafluorocyclobutane is also referred to as “DCHFCB.”

One embodiment of the composition of the disclosure is described in detail below.

A composition according to one embodiment of the disclosure contains CTFE and HFCB, and may further contain organic compounds other than CTFE and HFCB, or may further contain other components other than organic compounds. Examples of other organic compounds include DCHFCB, 1,1,2-trichloro-1,2,2-trifluoroethane (CFC-113), and trifluoroethylene (HFO-1123), and examples of components other than organic compounds include water, hydrogen, and hydrogen chloride.

The content of organic compounds with respect to the total amount of the composition is, for example, 25% by volume or more, or may be 30% by volume or more, 35% by volume or more, 40% by volume or more, 55% by volume or more, 70% by volume or more, 80% by volume or more, or 90% by volume or more.

### <CTFE>

The composition of the embodiment contains CTFE in a content of 8% by mass or more with respect to the total amount of organic compounds. The content of CTFE with respect to the total amount of organic compounds is 8% by mass or more, or may be 15% by mass or more, 25% by mass or more, 50% by mass or more, or 75% by mass or more. The upper limit of the CTFE content is not particularly limited. From the viewpoint of suppressing a decrease in the pH of the composition, the content of CTFE may be 90% by mass or less, or 85% by mass or less with respect to the total amount of organic compounds. The content of CTFE is preferably from 8% to 90% by mass, more preferably from 15% to 90% by mass, and still more preferably from 25% to 90% by mass with respect to the total amount of organic compounds.

In the embodiment, even when the CTFE content is equal to or greater than the above-described lower limit, by containing HFCB in an amount of 1 ppm by mass or more with respect to the total amount of organic compounds, the composition is suppressed from having a decrease in pH when left in contact with a metal for a long period of time. Furthermore, even when the CTFE content is equal to or less than the above-described upper limit, by containing HFCB in an amount of 1 ppm by mass or more with respect to the total amount of organic compounds, the composition is suppressed from having a decrease in pH when left in contact with a metal for a long period of time.

Here, the content of each component with respect to the total amount of organic compounds is determined by measurement using gas chromatography.

Specifically, a gas chromatograph (GC-7890A manufactured by Agilent Technologies Inc.) is used as a measuring device, a DB-1 column (manufactured by Agilent Technologies Inc.; length: 60 m; inner diameter: 250 μm; film thickness: 1 μm) is used as a column, and a flame ionization detector (FID) is used as a detector, and the measurement is performed under the following measurement conditions.
Injection temperature: 240 °C
Sample injection volume: 0.5 mL
Split ratio: 60/1
Linear speed: 35.8 cm/sec
Start of measurement: temperature of −30 °C, hold time of 10 minutes
Temperature increase rate: 10 °C/min
End of measurement: temperature of 240 °C, hold time of 20 minutes
Detection temperature: 250 °C.

The ratio of the mass of each component to the total mass of organic compounds detected by the above measurement is calculated and used as the content of each component.

### <HFCB>

The composition of the embodiment contains HFCB in a content of 1 ppm by mass or more with respect to the total amount of organic compounds. As described above, HFCB is a compound produced by the dimerization of CTFE and the elimination of chlorine.

The content of HFCB with respect to the total amount of organic compounds is 1 ppm by mass or more, and it is preferably 50 ppm by mass or more, more preferably 100 ppm by mass or more, still more preferably 2000 ppm by mass or more, and particularly preferably 3500 ppm by mass or more, from the viewpoint of suppressing a decrease in the pH of the composition. The upper limit of the HFCB content is not particularly limited. The content of HFCB may be 10,000 ppm by mass or less, or 5,000 ppm by mass or less with respect to the total amount of organic compounds. The content of HFCB is preferably from 1 to 10,000 ppm by mass, more preferably from 50 to 10,000 ppm by mass, and still more preferably from 100 to 10,000 ppm by mass with respect to the total amount of organic compounds.

The content of HFCB with respect to 100 parts by mass of CTFE is, for example, 0.03 parts by mass or more, preferably 0.05 parts by mass or more, more preferably 0.1 parts by mass or more, and still more preferably 0.3 parts by mass or more. The upper limit of the content of HFCB with respect to 100 parts by mass of CTFE is not particularly limited, and may be, for example, 3 parts by mass or less. The content of HFCB with respect to 100 parts by mass of CTFE is preferably from 0.03 to 3 parts by mass, more preferably from 0.05 to 3 parts by mass, still more preferably from 0.1 to 3 parts by mass, and particularly preferably from 0.3 to 3 parts by mass.

### <DCHFCB>

The composition of the embodiment preferably further contains DCHFCB. When the composition contains DCHFCB, the decrease in pH when the composition is left in contact with a metal for a long period of time is further suppressed. As mentioned above, DCHFCB is a compound produced by the dimerization of CTFE. Since this reaction is an equilibrium reaction, because the initial composition contains DCHFCB, the equilibrium is less likely to shift in the direction of further production of DCHFCB. It is presumed that the elimination of chlorine from DCHFCB is unlikely to occur as a result of the suppression of the production of DCHFCB, thereby suppressing the decrease in pH due to the production of hydrogen chloride.

The content of DCHFCB with respect to the total amount of organic compounds is preferably 1 ppm by mass or more, more preferably 10 ppm by mass or more, and still more preferably 50 ppm by mass or more, from the viewpoint of suppressing a decrease in the pH of the composition. The upper limit of the DCHFCB content is not particularly limited. The content of DCHFCB may be 1000 ppm by mass or less with respect to the total amount of organic compounds. The content of DCHFCB is preferably from 1 to 1000 ppm by mass, more preferably from 10 to 1000 ppm by mass, and still more preferably from 50 to 1000 ppm by mass with respect to the total amount of organic compounds.

The content of DCHFCB with respect to 100 parts by mass of CTFE is, for example, 0.03 parts by mass or more, preferably 0.05 parts by mass or more, and more preferably 0.1 parts by mass or more. The upper limit of the content of DCHFCB with respect to 100 parts by mass of CTFE is not particularly limited, and may be, for example, 0.5 parts by mass or less. The content of DCHFCB with respect to 100 parts by mass of CTFE is preferably from 0.03 to 0.5 parts by mass, more preferably from 0.05 to 0.5 parts by mass, and still more preferably from 0.1 to 0.5 parts by mass.

### <CFC-113>

The composition of the embodiment may further contain CFC-113. CFC-113 is a compound that serves as a raw material for producing CTFE. For example, as will be described later, by reacting CFC-113 with hydrogen, hydrogen atoms and chlorine atoms are eliminated from CFC-113, thereby obtaining CTFE and generating hydrogen chloride.

When the composition that contains CFC-113 is left in contact with a metal for a long period of time, hydrogen chloride is produced by the reaction of CFC-113, which may easily cause a decrease in the pH of the composition. Meanwhile, in the embodiment, since CTFE and HFCB are contained in the above-described contents, the generation of hydrogen chloride due to the dehydrochlorination reaction of CFC-113 is suppressed, and a decrease in the pH of the composition is also suppressed.

The content of CFC-113 with respect to the total amount of organic compounds is preferably 80% by mass or less, more preferably 75% by mass or less, still more preferably 50% by mass or less, even more preferably 40% by mass or less, particularly preferably 30% by mass or less, and extremely preferably 20% by mass or less, from the viewpoint of suppressing a decrease in the pH of the composition. The lower limit of the CFC-113 content is not particularly limited, and may be 0% by mass. The content of CFC-113 may be 0.1% by mass or more, or 1% by mass or more with respect to the total amount of organic compounds. The content of CFC-113 is preferably from 0% to 80% by mass, more preferably from 0.1% to 80% by mass, and still more preferably from 1% to 75% by mass with respect to the total amount of organic compounds.

### <HFO-1123>

The composition of the embodiment preferably further contains HFO-1123. HFO-1123 is a compound produced by the reaction of CTFE with hydrogen. When the composition contains HFO-1123, the decrease in pH when the composition is left in contact with a metal for a long period of time is further suppressed. The reason for this is unclear, but it is presumed that HFO-1123 is less likely to generate an acid due to dimerization or hydrolysis than CTFE.

The content of HFO-1123 with respect to the total amount of organic compounds is preferably 0.5% by mass or more, more preferably 0.9% by mass or more, still more preferably 1.5% by mass or more, particularly preferably 2.0% by mass or more, and extremely preferably 3.0% by mass or more, from the viewpoint of suppressing a decrease in the pH of the composition. The upper limit of the HFO-1123 content is not particularly limited. The content of HFO-1123 may be 50% by mass or less, or 40% by mass or less with respect to the total amount of organic compounds. The content of HFO-1123 is preferably from 0.5% to 50% by mass, more preferably from 0.9% to 50% by mass, and still more preferably from 0.9% to 40% by mass with respect to the total amount of organic compounds.

### <Water>

The composition of the embodiment may further contain water. Water may be contained in the composition in a gaseous state (i.e., water vapor) or in a liquid state. In a case in which the composition contains a large amount of water, the pH is more likely to decrease due to an acid such as hydrogen chloride contained in the composition. Meanwhile, in the embodiment, since the generation of an acid such as hydrogen chloride is suppressed by including HFCB in the above-described content, it is presumed that even when the composition contains water, a decrease in the pH of the composition is suppressed.

From the viewpoint of suppressing a decrease in pH, the water content with respect to the total amount of the composition is preferably 2.5% by mass or less (25,000 ppm by mass or less), more preferably 2.2% by mass or less (22,000 ppm by mass or less), and still more preferably 2.0% by mass or less (20,000 ppm by mass or less).

The lower limit of the water content is not particularly limited. The water content with respect to the total amount of the composition may be 5 ppm by mass or more, or 10 ppm by mass or more. In the process of producing the composition, water often gets mixed into the composition during the step of removing hydrogen chloride eliminated from CFC-113, and it is difficult to completely remove the mixed water. Meanwhile, in the composition of the embodiment, even when the water content is equal to or higher than the above-described lower limit, because the CTFE and HFCB contents are each within the ranges, a decrease in pH when the composition is left in contact with a metal for a long period of time is suppressed. Specifically, since the amount of hydrogen chloride generated by dimerization of CTFE and elimination of chlorine in the composition is small, it is presumed that even when the water content is equal to or greater than the above-described lower limit, the amount of hydrochloric acid generated, which causes a decrease in pH, is small, and therefore a decrease in the pH of the composition is unlikely to occur.

The water content with respect to the total amount of the composition is preferably from 5 to 25,000 ppm by mass, more preferably from 5 to 22,000 ppm by mass, and even more preferably from 10 to 22,000 ppm by mass.

The water content is determined by measuring the amount of water in the gaseous state produced using a Gastec Detector Tube (No. 6 for water vapor).

### <Method of Producing Composition>

The composition of the embodiment can be obtained, for example, by contacting a mixture containing CFC-113 and hydrogen with a catalyst so as to reduce CFC-113 with hydrogen.

When contacted with the catalyst, CFC-113 and hydrogen may be in the gas phase or liquid phase, but are preferably in the gas phase.

The contact of the mixture containing CFC-113 and hydrogen with the catalyst may be carried out, for example, by supplying CFC-113 and hydrogen separately to a reaction field in which the catalyst is present, or by supplying a mixture in which CFC-113 and hydrogen are mixed in advance.

The method of producing the composition of the embodiment may be a continuous method or a batch method. In a continuous production method, for example, the steps of supplying, as raw materials, CFC-113 and hydrogen to a reactor accommodating a catalyst, contacting the CFC-113 and hydrogen with the catalyst in the reactor, and removing the produced composition from the reactor are all carried out continuously.

The catalyst used in the method for producing the composition is preferably a catalyst containing copper and at least one of palladium or platinum, from the viewpoint of setting the contents of CTFE and HFCB within the respective above-described ranges. Hereinafter, a catalyst containing palladium and copper is also referred to as a “Pd—Cu catalyst,” and a catalyst containing platinum and copper is also referred to as a “Pt—Cu catalyst.”

The Pd—Cu catalyst may be a two-component catalyst composed of palladium and copper, a three-component catalyst composed of palladium, copper, and another component, or a catalyst containing four or more components. The Pd—Cu catalyst is preferably a two-component catalyst composed of palladium and copper, from the viewpoint of setting the contents of CTFE and HFCB within the respective above-described ranges.

Furthermore, the Pt—Cu catalyst may be a two-component catalyst composed of platinum and copper, a three-component catalyst composed of platinum, copper, and other components, or a catalyst containing four or more components. The Pt—Cu catalyst is preferably a two-component catalyst composed of platinum and copper, from the viewpoint of setting the contents of CTFE and HFCB within the respective above-described ranges.

The form of the catalyst is not particularly limited, and may be in the form of a powder, pellets, or spheres.

The specific surface area of the catalyst is, for example, from 0.1 to 1500 m²/g, and it is preferably from 1 to 1500 m²/g, more preferably from 100 to 1500 m²/g, from the viewpoint of setting the contents of CTFE and HFCB within the respective above-described ranges. The specific surface area is a value measured by the BET method (BET specific surface area).

From the viewpoint of setting the contents of CTFE and HFCB within the respective above-described ranges, the hydrogen content in the mixture is preferably from 0.8 to 10 times, more preferably from 0.9 to 10 times, still more preferably from 1 to 10 times, even more preferably from 1 to 5 times, particularly preferably from 1 to 2.5 times, and extremely preferably from 1 to 2 times the CFC-113 content on a volume basis.

The hydrogen content with respect to the total amount of the mixture is preferably from 44% to 91% by volume, more preferably from 50% to 91% by volume, and still more preferably from 52% to 91% by volume, from the viewpoint of setting the contents of CTFE and HFCB within the respective above-described ranges.

The content of CFC-113 with respect to the total amount of the mixture is preferably from 9% to 56% by volume, more preferably from 9% to 50% by volume, and still more preferably from 9% to 48% by volume, from the viewpoint of setting the contents of CTFE and HFCB within the respective above-described ranges.

The total content of CFC-113 and hydrogen with respect to the total amount of the mixture is preferably 80% by volume or more, more preferably 90% by volume or more, and still more preferably 95% by volume or more, from the viewpoint of setting the contents of CTFE and HFCB within the respective above-described ranges.

The temperature of the mixture when contacting the mixture with the catalyst, i.e., the reaction temperature is preferably from 150 °C to 300 °C, more preferably from 180 °C to 300 °C, and still more preferably from 190 °C to 300 °C, from the viewpoint of setting the contents of CTFE and HFCB within the respective above-described ranges.

The pressure at which the mixture is contacted with the catalyst is preferably ordinary pressure (i.e., atmospheric pressure).

The time for contacting the mixture with the catalyst, i.e., the reaction time, is preferably from 0.5 to 100 seconds, more preferably from 1 to 75 seconds, and still more preferably from 4 to 75 seconds, from the viewpoint of setting the contents of CTFE and HFCB within the respective above-described ranges.

When the method of producing the composition is a continuous method, the reaction time (sec) is calculated, for example, using the following formula.

Reaction time (sec)=[length of catalyst packed in reactor (cm)]/[linear velocity (cm/sec)]

The linear velocity refers to the length that the mixture passes through the catalyst per unit time.

The method of producing the composition of the embodiment is not limited to the above-described method, and, for example, the contents of CTFE and HFCB may be set within the respective above-described ranges by adding HFCB to a composition containing CTFE.

### [Composition-Containing Container]

A composition-containing container according to one embodiment of the disclosure includes: the composition described above; and a container that accommodates the composition, in which a member that contacts the composition is at least partially formed of a metal material.

Examples of the above-described composition-containing container may include a container which is entirely formed of a metal material, a container having a multi-layer structure with the innermost layer formed of a metal material, and a container having a coating formed of a metal material in the area that contacts the composition.

The metal material is preferably, for example, a metal selected from the group consisting of iron, copper, aluminum, stainless steel, titanium, nickel, zinc, tin, brass, magnesium, chromium, lead, silver, tungsten, niobium, molybdenum, and tantalum; an alloy containing at least one metal selected from the above group of metals; or a compound containing at least one metal selected from the above group of metals.

Examples of alloys include nickel-chrome plating, solder, and tin plating.

Examples of metal-containing compounds include alumite sulfate, zinc phosphate, and iron phosphate.

The container may include a container body and a portion that contacts the composition, and the container body and the portion may be formed of different materials. Examples of materials forming the container body include iron (steel), stainless steel, carbon steel, manganese steel, chromium-molybdenum steel, other low-alloy steels, aluminum alloys, and glass. Examples of stainless steel include SUS316, SUS304, and JFE443CT.

The shape and size of the container can be designed to suit the purpose. Examples include a storage tank, which is a fixed storage container, a transportable container, and a shipping container. Examples of a transportable container include a 1-L glass bottle, a 20-L pail, a 200-L drum, a ton container, an aerosol can, and a high-pressure gas container (non-refillable container, welded container, seamless container, or the like). Examples of a shipping container include a tanker truck, an ISO container, and a self-loader.

The container may be sealable. The method of sealing the container is not particularly limited, and examples thereof include a method of sealing with a screw cap and a method of sealing with a valve. An elastic member, such as a cushioning material or a sealing material, may be used to improve airtightness.

Examples of materials for the elastic member include styrene-butadiene rubber (SBR), isoprene rubber (IR), butadiene rubber (BR), ethylene-propylene rubber (EPM), urethane rubber (U), chloroprene rubber (CR), nitrile rubber (NBR), hydrogenated nitrile rubber (HNBR), fluorine rubber (FKM) in which carbon-hydrogen bonds exist in part of the main chain, perfluoroelastomer in which the main chain is completely fluorinated, chlorinated polyethylene (CM), acrylic rubber (ACM), polysulfide rubber (T), epichlorohydrin rubber, styrene-based thermoplastic elastomer, olefin-based thermoplastic elastomer, ester-based thermoplastic elastomer, urethane-based thermoplastic elastomer, amide-based thermoplastic elastomer, PVC-based thermoplastic elastomer, and fluorine-based thermoplastic elastomer.

### [System]

A system according to one embodiment of the disclosure includes: the above-described composition; and a flow path that allows the composition to pass therethrough, in which a member that contacts the composition is at least partially formed of a metal material. The system may further include other members if necessary.

The flow path included in the system may be, for example, a flow path which is entirely formed of a metal material, a flow path having a multi-layer structure with the innermost layer formed of a metal material, or a flow path having a coating formed of a metal material in the area that contacts the composition.

The details of the metal material forming at least part of the flow path are the same as those of the metal material forming at least part of the container described above.

The flow path may include a flow path body and a portion that contacts the composition, and the flow path body and the portion may be formed of different materials. Examples of materials forming the flow path body include iron (steel), stainless steel, carbon steel, manganese steel, chromium-molybdenum steel, other low-alloy steels, aluminum alloys, and glass. Examples of stainless steel include SUS316, SUS304, and JFE443CT.

The shape and size of the flow path can be designed to suit the purpose.

Examples of the system of the embodiment include a production apparatus used in the method of producing the above-described composition, and a production apparatus for producing other compounds using the above-described composition as a raw material.

An example of a production apparatus used in the method of producing the composition described above is an apparatus including: a reactor that accommodates a catalyst, in which a mixture containing, as raw materials, CFC-113 and hydrogen is supplied to the catalyst and reacted therewith; a collection vessel that collects the composition produced by the reaction; and a flow path that supplies the composition from the reactor to the collection vessel. The above-described apparatus may further include a hydrogen chloride trap for separating hydrogen chloride from a product produced by the reaction, and a dehydration device for removing water from the product. In the production apparatus, as the flow path that supplies the composition from the reactor to the collection vessel, a flow path in which a member that contacts the composition is at least partially formed of a metal material is applied. The above-described composition-containing container may be used as the collection vessel.

An example of a production apparatus for producing a different compound using the above-described composition as a raw material is an apparatus including: a raw-material-accommodating container that accommodates the composition as a raw material; a reactor that reacts the composition as a raw material; and a flow path that supplies the composition from the raw-material-accommodating container to the reactor. In the production apparatus, as the flow path that supplies the composition from the raw-material-accommodating container to the reactor, a flow path in which a member that contacts the composition is at least partially formed of a metal material is applied. The above-described composition-containing container may be used as the raw-material-accommodating container.

### EXAMPLES

Hereinafter, the disclosure will be described more specifically with reference to examples. The disclosure is not limited to the following examples as long as it does not depart from the gist of the disclosure.

### Example 1

### <Production of Composition>

A Pt—Cu catalyst was used as the catalyst. The Pt—Cu catalyst used was a two-component catalyst composed of platinum and copper, and the specific surface area of the catalyst was 1100 m²/g.

The catalyst was packed into a 20ASUS tubular reactor such that the catalyst length was 30 cm.

A 1.5/1 (mol/mol) mixed gas of hydrogen and CFC-113 was allowed to pass through the catalyst in the reactor so as to carry out a hydrogen reduction reaction of CFC-113, and the resulting product was passed through an alkali trap, thereby obtaining an initial composition.

The reaction conditions for the hydrogen reduction reaction were a raw material load of 675 kg/m3/hr, a reaction temperature of 250 °C, a reaction time of 9.3 seconds, and atmospheric pressure inside the reactor. The initial composition thus obtained corresponds to an example of the composition of the disclosure.

The initial composition thus obtained was filled into a 25-cc stainless steel cylinder container so as to prepare a composition-containing container, which was then placed in a thermostatic chamber at 70 °C and left to stand for 72 hours, thereby obtaining a composition after standing.

The content of each component with respect to the total amount of organic compounds was analyzed by gas chromatography using the method described above for each of the initial composition and the composition after standing. The results are shown in Table 1.

Furthermore, the content of water with respect to the total amount of the composition was measured for each of the initial composition and the composition after standing by the method described above. The results are shown in Table 1. In Table 1, “-” means that the measurement was omitted. The content of organic compounds with respect to the total amount of the initial composition was 25% by volume.

Furthermore, the initial composition and the composition after standing were separately allowed to pass through a plastic bottle containing ion-exchanged water, and the pH of each resulting aqueous solution was measured with a pH meter. The amount of change in pH of the composition after standing with respect to the pH of the initial composition (the pH of the composition after standing—the pH of the initial composition) was determined. The results are shown in Table 1. In Table 1, negative values for the amount of change in pH mean that the pH decreased after being left for 72 hours.

### Example 2

An initial composition and a composition after standing were obtained in the same manner as in Example 1, except that the reaction temperature was changed to 200 °C and the reaction time was changed to 10.3 seconds.

For the initial composition thus obtained and the composition after standing, the content of each component with respect to the total amount of organic compounds, the content of water with respect to the total amount of the composition, and the amount of change in pH were determined in the same manner as in Example 1. The results are shown in Table 1. The content of organic compounds with respect to the total amount of the initial composition was 35% by volume.

### Example 3

An initial composition and a composition after standing were obtained in the same manner as in Example 1, except that the reaction temperature was changed to 180 °C and the reaction time was changed to 10.8 seconds.

For the initial composition thus obtained and the composition after standing, the content of each component with respect to the total amount of organic compounds, the content of water with respect to the total amount of the composition, and the amount of change in pH were determined in the same manner as in Example 1. The results are shown in Table 1. The content of organic compounds with respect to the total amount of the initial composition was 38% by volume.

TABLE 1

| | | Example 1 | | Example 2 | | Example 3 | |
|---|---|---|---|---|---|---|---|
| | | | After | | After | | After |
| | | Initial | standing | Initial | standing | Initial | standing |
| Organic | CFC-113 | 13.465 | 13.652 | 72.971 | 67.306 | 92.355 | 84.430 |
| compound | CTFE | 78.682 | 77.814 | 25.116 | 30.224 | 7.106 | 14.468 |
| composition | HFO- | 3.097 | 3.207 | 0.946 | 1.204 | 0.252 | 0.570 |
| (% by | 1123 | | | | | | |
| mass) | HFCB | 0.3810 | 0.7690 | 0.015 | 0.1650 | 0.0000 | 0.0715 |
| | DCHFCB | 0.0990 | 0.1000 | 0.0000 | 0.0000 | 0.0000 | 0.0000 |

| | | | | | | |
|---|---|---|---|---|---|---|
| Water content with | 1.34 | — | 1.34 | — | 2.54 | — |
| respect to the total | | | | | | |
| amount of the | | | | | | |
| composition | | | | | | |
| (% by mass) | | | | | | |

| | | | |
|---|---|---|---|
| Change in pH | −1.35 | −1.85 | −2.14 |

Examples 1 and 2 are working examples, and Example 3 is a comparative example.

As shown in Table 1, it was found that the initial compositions of Examples 1 and 2 suppressed a decrease in pH even when left in contact with a metal for a long period of time. Furthermore, in the initial composition of Example 1, the decrease in pH was further suppressed compared to the initial composition of Example 2.

The disclosure of Japanese Patent Application No. 2023-205627, filed on Dec. 5, 2023, is incorporated herein by reference in its entirety. Furthermore, all publications, patent applications, and technical standards mentioned herein are incorporated by reference herein to the same extent as if each individual publication, patent application, and technical standard were specifically and individually indicated to be incorporated by reference.

1. A composition, comprising chlorotrifluoroethylene and hexafluorocyclobutene, wherein:
a content of the chlorotrifluoroethylene is 8% by mass or more with respect to a total amount of organic compounds included in the composition, and
a content of the hexafluorocyclobutene is 1 ppm by mass or more with respect to the total amount of organic compounds included in the composition.

2. The composition according to claim 1, wherein the content of the hexafluorocyclobutene is 50 ppm by mass or more with respect to the total amount of organic compounds included in the composition.

3. The composition according to claim 1, further comprising 1,2-dichloro-1,2,3,3,4,4-hexafluorocyclobutane,
wherein a content of the 1,2-dichloro-1,2,3,3,4,4-hexafluorocyclobutane is 1 ppm by mass or more with respect to the total amount of organic compounds included in the composition.

4. The composition according to claim 1, further comprising 1,1,2-trichloro-1,2,2-trifluoroethane.

5. The composition according to claim 3, further comprising 1,1,2-trichloro-1,2,2-trifluoroethane.

6. The composition according to claim 1, further comprising trifluoroethylene.

7. The composition according to claim 6, wherein a content of the trifluoroethylene is 0.3% by mass or more with respect to the total amount of organic compounds included in the composition.

8. The composition according to claim 1, further comprising water,
wherein a content of the water is 5 ppm by mass or more with respect to a total amount of the composition.

9. A composition-containing container, comprising:
the composition according to claim 1; and
a container that accommodates the composition, in which a member that contacts the composition is at least partially formed of a metal material.

10. A system, comprising:
the composition according to claim 1; and
a flow path that allows the composition to pass therethrough, in which a member that contacts the composition is at least partially formed of a metal material.

11. A method of producing the composition according to claim 1, the method comprising:
contacting a mixture comprising 1,1,2-trichloro-1,2,2-trifluoroethane and hydrogen with a catalyst comprising copper and at least one of palladium or platinum.

12. The method of producing a composition according to claim 11, wherein a temperature of the mixture when contacting the mixture with the catalyst is from 150 °C to 300 °C.

13. The method of producing a composition according to claim 11, wherein the catalyst is a two-component catalyst composed of copper and either palladium or platinum.

14. The method of producing a composition according to claim 11, wherein a content of hydrogen in the mixture is from 1 to 2.5 times a content of the 1,1,2-trichloro-1,2,2-trifluoroethane on a volume basis.

15. The composition according to claim 3, further comprising trifluoroethylene.

16. The composition according to claim 15, wherein a content of the trifluoroethylene is 0.3% by mass or more with respect to the total amount of organic compounds included in the composition.

17. A method of producing the composition according to claim 3, comprising:
contacting a mixture comprising 1,1,2-trichloro-1,2,2-trifluoroethane and hydrogen with a catalyst comprising copper and at least one of palladium or platinum.

18. The method of producing a composition according to claim 17, wherein a temperature of the mixture when contacting the mixture with the catalyst is from 150 °C to 300 °C.

19. The method of producing a composition according to claim 17, wherein the catalyst is a two-component catalyst composed of copper and either palladium or platinum.

20. A composition-containing container, comprising:
the composition according to claim 3; and
a container that accommodates the composition, in which a member that contacts the composition is at least partially formed of a metal material.
